(19) United States
(12) Patent Application Publication
(10) Pub. No.: US 2013/0004384 A1
(43) Pub. Date: Jan. 3, 2013

(54) STERILIZING APPARATUS AND METHOD USING HYDROGEN PEROXIDE

(76) Inventor: Chul Won Yoo, Seoul (KR)

(21) Appl. No.: 13/635,466

(22) PCT Filed: Mar. 16, 2011
(86) PCT No.: PCT/KR2011/001847

§ 371 Date: Sep. 17, 2012

(30) Foreign Application Priority Data
Mar. 17, 2010 (KR) 10-2010-0023705

(51) Int. Cl. A61L 2/18

(52) U.S. Cl. 422/292

(57) ABSTRACT

Disclosed is a sterilization apparatus using hydrogen peroxide, which comprises a housing which has a sterilization chamber for accommodating to-be-sterilized objects; an evaporator connected with the sterilization chamber for supplying hydrogen peroxide vapor to the sterilization chamber for the purpose of sterilizing to-be-sterilized objects; a hydrogen peroxide supply apparatus connected with the evaporator for the purpose of supplying liquid hydrogen peroxide to the evaporator; an enrichment chamber connected with the evaporator for the purpose of evaporating moisture from the hydrogen peroxide supplied to the evaporator and sucking the evaporated moisture, thus enriching the hydrogen peroxide; a vacuum pump connected with the enrichment chamber for the purpose of providing a vacuum pressure to the enrichment chamber; a blower which includes a fan disposed in the interior of the sterilization chamber for forcibly flowing the air of the sterilization chamber and is a motor for driving the fan; and a fan heater disposed in the interior of the sterilization chamber for the purpose heating the air flowing by means of the fan. The sterilization apparatus using hydrogen peroxide according to the present invention makes it possible to enrich the hydrogen peroxide using an enrichment chamber, and the sterilization power with respect to the to-be-sterilized objects can be enhanced.

### TECHNICAL FIELD

The present invention relates to an apparatus for sterilizing a to-be-sterilized object using a chemical sterilant, and particularly to a sterilization apparatus and method using a hydrogen peroxide which makes it possible to sterilize a to-be-sterilized object using a hydrogen peroxide.

### BACKGROUND ART

In the sterilization on medical tools, different from a cleaning or disinfection, it means that all kinds of living microorganisms are fully sterilized by way of physical, chemical operations, so the sterilization means a high level treatment. The medical tools are sterilized using high temperature dry heat or is the heat from vapor or in such a way that chemicals such as formaldehyde or ethylene oxide are used in a gas or vapor state.

A plurality of different medical tools such as a fiber optical device, an endoscope, a certain motored tool, etc. are quite weak to heat or moisture or both of them, so the conventional sterilization method using heat cannot be actually adapted. The method of using formaldehyde or ethylene oxide gas is a toxic gas with potential cancer-causing factors, so a lot of attentions are needed when handling.

In particular, when ethylene oxide gas is used, an aeration time of more than 8 hours is needed so as to eliminate the gas from the sterilized products, which becomes a huge factor consequently prolonging sterilization cycle time. In terms of the installation environment and management, an independent space and the provision of a gas detector are needed, the work becomes complicated.

In addition to a sterilization method of using ethylene oxide gas of formaldehyde gas, there is a sterilization method of using plasma. In a conventional sterilization apparatus using plasma, when hydrogen peroxide is injected in a plasma state, the pressure rises, which makes it hard to generate plasma, and when plasma is generated in a state that hydrogen peroxide is injected, the plasma comes to decompose hydrogen peroxide. So, the plasma process is performed before or after the injection of the hydrogen peroxide, which means the sterilization by means of hydrogen peroxide rather than plasma.

In the conventional sterilization method using plasma, since the to-be-sterilized object and the plasma are likely to contact directly each other, so the medical tools made of polymer materials discolor or change the natures such as a physical hardness. In addition, since the plasma is needed to generate uniformly in the reaction container, the size of the reaction contained is limited, and if the reaction container has a lot of objects to be sterilized, the sterilization cannot be properly performed.

When sterilizing the to-be-sterilized objects using hydrogen peroxide, the handling, transportation and use of a high concentration hydrogen peroxide with higher than 60% of concentration is prohibited in compliance with the danger and safety management law, so 50˜58% of hydrogen peroxide is used. In this case, a desired sterilization power cannot be obtained. In order to improve the above mentioned problems, a vapor sterilization method is developed. The vapor sterilization method is directed to enhancing sterilization power in such a way to heating and vaporizing hydrogen peroxide solution.

The conventional vapor sterilization method is directed to generating hydrogen peroxide in a form of plasma inside or outside the reaction container and spreading it to contact with the to-be-sterilized objects, so the sterilization process is performed under a low pressure vacuum environment. The above mentioned conventional sterilization method is performed using a lumen the diameter of which is less than 1 mm and the length of which is above 50 cm in a flexible endoscope; however it features in that the penetration of the hydrogen peroxide which is a key function in terms of sterilization is too weak.

In other words, since water has a vapor pressure higher than hydrogen peroxide, it quickly vaporizes, and since the molecular weight of water is low, water can vaporize and spread more quickly than hydrogen peroxide. So, the vapor of water spreads quickly into a thin, long limited region while limiting the injection of hydrogen peroxide vapor, so the sterilization efficiency with respect to the to-be-sterilized objects becomes low.

In terms of the sterilization method using hydrogen peroxide and plasma, it is true that the temperature of the sterilization chamber and the temperature of the to-be-sterilized object affect the sterilization efficiency; however in the conventional low temperature sterilizer using a temperature lower than 60° C., a low temperature plasma might be used in the vacuum state or a more passive method of using an internal radiation heat is used while maintaining the sterilization chamber to be lower than 60° C. in the outside. In a conventional way, the hospitals feature in that the to-be-sterilized objects are washed, and waters are removed, and then the sterilization is conducted using the sterilizer. The conventional plasma and hydrogen peroxide sterilization apparatus frequently causes incomplete sterilization, so re-sterilization processes are somehow needed.

The incomplete sterilization occurs because when the temperature of sterilization lowers in the sterilization chamber due to the to-be-sterilized objects, the vaporized hydrogen peroxide solution does not deeply penetrate the complicated to-be-sterilized objects having a spread limiting region such as long, narrow lumen and becomes condensed, so even though too much amount of hydrogen peroxide is injected, the vaporizing amount becomes quite limited when the temperature is low.

In the conventional sterilization method and sterilization apparatus using a low temperature plasma and hydrogen peroxide do not have dry functions. So, the sterilized objected are dried using a separate dryer, which causes many inconveniences, and the whole work time is prolonged.

Due to the above mentioned many problems in the conventional art, there are still needs on a more effective method which makes it possible to efficiently sterilize, at low temperatures, various kinds of to-be-sterilized objects to which vapor cannot reach well and which have long and narrow regions and have closed ends and which makes it possible sterilize at a low temperature.

### DISCLOSURE OF INVENTION

Accordingly, the present invention is made to improve the above mentioned problems, and it is an object of the present invention to provide a sterilization apparatus and method using hydrogen peroxide which make it possible to diversify the processes for the purpose of adjusting a sterilization time and a sterilization degree matching with each to-be-sterilized object, thus greatly enhancing the sterilization efficiency and conveniences when in use with respect to the different items and the different to-be-sterilized objects each having narrow, long regions.

To achieve the above objects, there is provided a sterilization apparatus using hydrogen peroxide, comprising a housing which has a sterilization chamber for accommodating to-be-sterilized objects; an evaporator connected with the sterilization chamber for supplying hydrogen peroxide vapor to the sterilization chamber for the purpose of sterilizing to-be-sterilized objects; a hydrogen peroxide supply apparatus connected with the evaporator for the purpose of supplying liquid hydrogen peroxide to the evaporator; an enrichment chamber connected with the evaporator so as to evaporate moisture from the hydrogen peroxide supplied to the evaporator and to suck the evaporated moisture and to enrich the hydrogen peroxide; a vacuum pump connected with the enrichment chamber for the purpose of providing a vacuum pressure to the enrichment chamber; a blower which includes a fan disposed in the interior of the sterilization chamber for forcibly flowing the air of the sterilization chamber and a motor for driving the fan; and a fan heater disposed in the interior of the sterilization chamber for the purpose heating the air flowing by means of the fan.

The sterilization apparatus using hydrogen peroxide according to an embodiment of the present invention further comprises an enrichment adjusting valve disposed between the evaporator and the enrichment chamber for thereby opening and closing the flow passage between the evaporator and the enrichment chamber.

The sterilization apparatus using hydrogen peroxide according to an embodiment of the present invention further comprises an enrichment chamber pressure release valve connected to the enrichment chamber for the purpose of introducing external air into the interior of the enrichment chamber.

The vacuum pump is connected with the sterilization chamber for the purpose of providing vacuum pressure to the sterilization chamber, and between the sterilization chamber and the vacuum pump is disposed a sterilization chamber pressure valve for the purpose of opening and closing the flow passages between the sterilization chamber and the vacuum pump.

The sterilization apparatus using hydrogen peroxide according to an embodiment of the present invention further comprises a sterilization chamber pressure release valve connected to the sterilization chamber for the purpose of introducing external air into the interior of the sterilization chamber.

The sterilization apparatus using hydrogen peroxide according to an embodiment of the present invention further comprises an evaporator heater for heating the evaporator and an enrichment chamber heater for the purpose of heating the enrichment chamber.

The volume of the enrichment chamber is in a range of 10 ml˜the volume of the sterilization chamber.

The sterilization apparatus using hydrogen peroxide according to an embodiment of the present invention further comprises an enrichment chamber pressure meter connected with the enrichment chamber for the purpose of detecting the pressure of the enrichment chamber.

The fan is disposed closer to a side wall of one side of the housing, and the motor is disposed at an outer side of the side wall, and a rotary shaft of the motor passes through the side wall and is connected with the fan.

The fan heater is disposed between the fan and the side wall.

At the fan is installed a guide plate which faces the fan at the front side of the fan in the interior of the sterilization chamber and which rotates in a rotation direction that the air flowing to the fan gathers at the center of the fan, and the air of the interior of the sterilization chamber collides with the side wall by way of the fan and the fan heater and flows to the front side of the fan by way of the space between the guide plate and the inner surface of the housing.

In the sterilization apparatus using hydrogen peroxide, there is further provided a sterilization chamber temperature sensor disposed at the passage for detecting the temperature of the interior of the sterilization chamber.

In the sterilization apparatus using hydrogen peroxide, there are further provided a photo catalyst disposed in the interior of the sterilization chamber; and a violet ray generator which has a violet ray lamp disposed in the interior of the sterilization chamber for emitting violet ray to the photo catalyst.

The photo catalyst is coated on the surface of the fan.

The photo catalyst contains TiO₂.

To achieve the above objects, there is provided a sterilization method using hydrogen peroxide comprising (a) inputting a to-be-sterilized object into the interior of the sterilization chamber; (b) raising the temperature of the air and the to-be-sterilized object in the interior of the sterilization chamber by operating a fan and a fan heater installed in the interior of the sterilization chamber; (c) supplying liquid hydrogen peroxide to the evaporator connected with the sterilization chamber; (d) reducing the pressure of the enrichment chamber connected with the evaporator to be lower than the atmospheric pressure; (e) opening the flow passage between the evaporator and the enrichment chamber, evaporating the moisture from the hydrogen peroxide supplied to the evaporator, sucking the evaporated vapor into the enrichment chamber and enriching the hydrogen peroxide; (f) closing the flow passage between the evaporator and the enrichment chamber when the pressure of the enrichment chamber rises as the moisture evaporated from the hydrogen peroxide is introduced into the enrichment chamber; (g) opening the flow passage between the sterilization chamber and the evaporator and supplying the hydrogen peroxide into the sterilization chamber; and (h) operating the fan and the fan heater installed in the interior of the sterilization chamber and forcibly flowing the air of the interior of the sterilization chamber while heating it.

The sterilization method using hydrogen peroxide according to an embodiment of the present invention is implemented as the routines from the step (d) to the step (f) are repeatedly performed before the step (g).

There is further provided a step for heating the evaporator and the enrichment chamber before or after the step (c), and the temperatures of the evaporator and the enrichment chamber are in a range of 30° C.˜150° C.

There are further provided a step for lowering the pressure of the sterilization chamber to be lower than the atmospheric pressure before the step (g), and a step for making the pressure of the sterilization chamber same as the atmospheric pressure by supplying an external air to the sterilization chamber.

The sterilization method using hydrogen peroxide according to an embodiment of the present invention further comprises a step for emitting violet ray to the photo catalyst disposed in the interior of the sterilization chamber before or after the step (g).

### Advantageous Effects

The sterilization apparatus using hydrogen peroxide according to the present invention makes it possible enhance hydrogen peroxide by vaporizing moisture from the hydrogen peroxide from the evaporator by lowering the pressure of the enrichment chamber connected to the evaporator, for thereby enhancing sterilization power with respect to the to-be-sterilized objects accommodated in the sterilization chamber because the enriched hydrogen peroxide is supplied from the evaporator to the sterilization chamber.

In addition, a sterilization environment proper to different to-be-sterilized objects can be formed by adjusting the amount of hydrogen peroxide depending on each turn in such a way to adjust the number of vaporization of moisture with respect to hydrogen peroxide. So, it is possible to enhance sterilization efficiency with respect to a narrow, long item such as an endoscope and a to-be-sterilized object one side of which is closed like lumen.

The sterilization time can be reduced by diversifying the processes for the purpose of adjusting the process time and the sterilization power to match with each to-be-sterilized object by properly adjusting the concentration of hydrogen peroxide, and it is possible to prevent the service life of the to-be-sterilized object from reducing because of sterilization.

The sterilization apparatus using hydrogen peroxide according to the present invention is directed to quickly raising the temperatures of the to-be-sterilized objects in the interior of the sterilization chamber by means of the convention phenomenon of warm wind using a fan and a heater, and the temperature of the air in the sterilization chamber and the to-be-sterilized objects can be raised uniformly. So, when the hydrogen peroxide vaporizes, the moisture first penetrates into a long, narrow spread limit region, thus preventing the condensation, and the hydrogen peroxide does not condense, and more hydrogen peroxide can vaporize, thus more enhancing the sterilization performance.

In addition, the sterilization apparatus using hydrogen peroxide according to the present invention is directed to converting the oxygen, moisture in the air and the injected hydrogen peroxide into plasma states, and they are circulated using the fan, thus producing high density OH radicals. So, expensive plasma can be produced at a low cost, and the sterilization performance can be is enhanced more.

The sterilization apparatus using a hydrogen peroxide according to the present invention is directed to vacuuming after the air and the to-be-sterilized objects in the interior of the sterilization chamber are fully heated sing a heated air convection phenomenon generated with the aid of the fan and the heater, so the moisture can be quickly eliminated from the to-be-sterilized and the sterilization chamber, so the to-be-sterilized objects can be fully dried without using a separate drier. Since the separate drier is not needed in the present invention, the facility cost can be saved.

### BRIEF DESCRIPTION OF DRAWINGS

FIG. 1 is a schematic view illustrating a construction of a sterilization apparatus using hydrogen peroxide according to an embodiment of the present invention.

FIG. 2 is a schematic block diagram illustrating a sterilization apparatus using hydrogen peroxide according to an embodiment of the present invention.

FIG. 3 is a view illustrating a pressure change of an enrichment chamber in the course of sterilization by using a sterilization apparatus using hydrogen peroxide according to an embodiment of the present invention.

FIG. 4 is a graph of an internal pressure change of an evaporator and an enrichment chamber based on the repeat of the enrichment procedure in a sterilization experiment using a sterilization apparatus using hydrogen peroxide according to an embodiment of the present invention.

FIG. 5 is a graph of a comparison of a temperature change of a sterilization time as time passes when a fan and a fan heater are used and when they are not used.

FIG. 6 is a view illustrating a result of the hydrogen peroxide concentration in the sterilization chamber based on the temperature in the sterilization chamber and the concentration of hydrogen peroxide.

FIG. 7 is a graph showing a result of the measurement performed every 10 seconds with respect to the change in the concentration of hydrogen peroxide by comparing the results before and after the fan and the violet ray generator are operated.

### DESCRIPTIONS OF THE REFERENCE NUMERALS

10: sterilization apparatus 11: sterilization chamber
12: housing 13: evaporator
14: hydrogen peroxide supplier 15: enrichment apparatus
16: vacuum pump 17: blower
18: violet ray generator 19: door
20: hydrogen peroxide vapor supply adjusting valve
21: sterilization chamber pressure adjusting valve
22: sterilization chamber pressure release valve
23: sterilization chamber pressure meter
24: fan heater 25, 26: first and second sterilization chamber heaters
30: hydrogen peroxide supply adjusting valve 31: evaporator heater
33: enrichment chamber 34: enrichment adjusting valve
35: enrichment chamber pressure adjusting valve
36: enrichment chamber pressure release valve
37: enrichment chamber pressure meter
38: enrichment chamber heater
40: fan 41: motor
42: photo catalyst 43: guide plate
45: violet ray lamp 47: controller

### BEST MODES FOR CARRYING OUT THE INVENTION

Hereinafter, the sterilization apparatus and method using hydrogen peroxide according to an embodiment of the present invention will be described.

During the descriptions of the present invention, the size and shape of the elements shown in the drawings might be seen over emphasized for a clear, convenient and simplified construction. In addition, the terms defined in consideration of the construction and operation of the present invention might differ from the operator's intention or practices. The terms should be interpreted as the meaning and concept matching with the technical concepts of the present invention based on the contents over the specifications of the present invention.

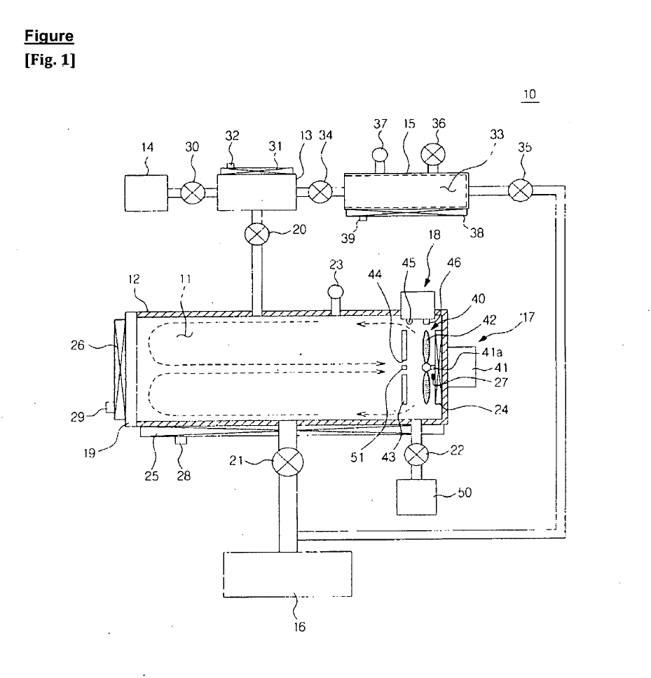

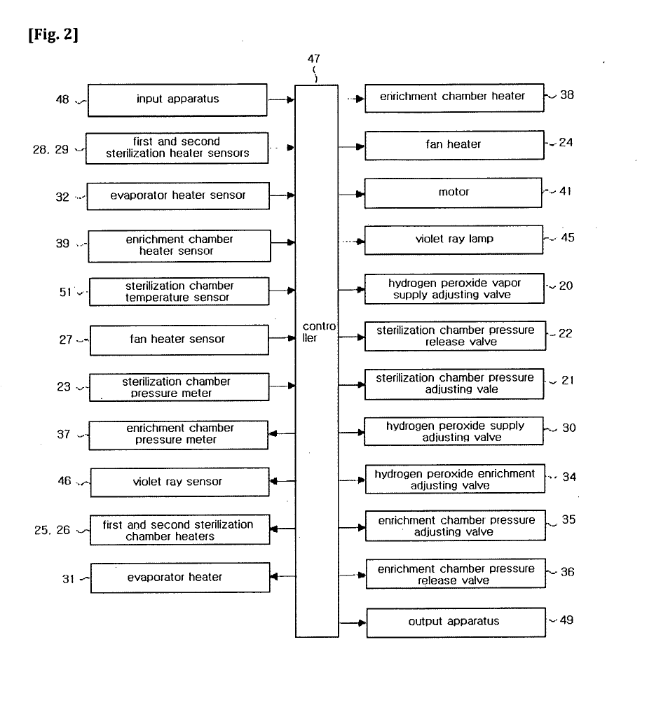

FIGS. 1 and 2 are schematic views illustrating a sterilization apparatus using hydrogen peroxide according to an embodiment of the present invention.

As shown in FIG. 1, the sterilization apparatus 10 using hydrogen peroxide according to an embodiment of the present invention comprises a housing 12 with a sterilization chamber 11 filled with a to-be-sterilized object, an evaporator 13 configured to supply hydrogen peroxide vapor to the sterilization chamber 11, a hydrogen peroxide supply apparatus 14 configured to supply hydrogen peroxide to the evaporator 13, an enrichment apparatus 15 for enriching the hydrogen peroxide supplied to the evaporator 13, a vacuum pump 16 configured to provide a vacuum pressure, a blower 17 configured to forcibly flow the air in the interior of the sterilization chamber 11, and a violet ray generator 18 configured to emit violet ray. At one side of the housing 12 is engaged a door 19 for opening and closing the opening which is used as a doorway for the sake of the to-be-sterilized objects.

The sterilization chamber 11 is connected with the evaporator 13 and the vacuum pump 15. Between the sterilization chamber 11 and the evaporator 13 is installed the hydrogen peroxide vapor supply adjusting valve 20, and between the chamber 11 and the vacuum pump 16 is installed the sterilization chamber pressure adjusting valve 21. In addition, at one side of the sterilization chamber 11 is connected a sterilization chamber pressure release valve 22 for introducing external air into the sterilization chamber 11. The hydrogen peroxide vapor supply adjusting valve 20, the sterilization chamber pressure adjusting valve 21 and the sterilization chamber pressure release valve 22 are controlled by means of the controller 47.

The hydrogen peroxide vapor supply adjusting valve 20 is configured to adjust the supply of the hydrogen peroxide vapor with respect to the sterilization chamber 11 as the passage connecting the sterilization chamber 11 and the evaporator 13 are closed and opened. The sterilization chamber pressure adjusting valve 21 is configured to open and close the flow passage between the sterilization chamber 11 and the vacuum pump 15. When the vacuum pump 15 starts operating in a state that the sterilization chamber pressure adjusting valve 21 is open, the air of the sterilization chamber 11 is discharged to the vacuum pump 15, and the inner pressure of the sterilization chamber 11 lowers.

The sterilization chamber pressure release valve 22 is configured to introduce the external air into the interior of the sterilization chamber 11, and when the sterilization chamber pressure release valve 22 is open, the external air is introduced into the sterilization chamber 11, and the inner pressure of the sterilization chamber 11 rises. The internal pressure of the sterilization chamber 11 is detected by means of the sterilization chamber pressure meter 23 installed in the housing 12, and the controller 47 receives a detection signal of the sterilization camber pressure meter 23 and controls the operations of the vacuum pump 15, the sterilization chamber pressure adjusting valve 21, the sterilization chamber pressure release valve 22, thus properly adjusting the internal pressure of the sterilization chamber 11.

The internal temperature of the sterilization chamber 11 is adjusted by the fan heater 24 installed in the interior of the sterilization chamber 11, the first sterilization chamber heater 25 installed in the housing 12, and the second sterilization camber heater 26 installed at the door 19. The heaters 24, 25 and 26 are controlled by means of the controller 47. The controller 47 serves to receive the signals from the first sterilization chamber heater sensor 28, the second sterilization chamber heater sensor 29 and the sterilization chamber temperature sensor 51 and controls the heaters 24, 25 and 26 for thereby constantly maintaining the temperature of the sterilization chamber 11.

As shown in FIG. 1, the evaporator 13 is connected with the hydrogen peroxide supply apparatus 14. Between the evaporator 13 and the hydrogen peroxide supply apparatus 14 is installed a hydrogen peroxide supply adjusting valve 30 which is controlled by the controller 47. The hydrogen peroxide supply apparatus 14 serves to supply liquid hydrogen peroxide to the evaporator 13. The temperature of the evaporator 13 is adjusted by means of the evaporator heater 31 controlled by means of the controller 47. The controller 47 serves to constantly maintain the temperature of the evaporator 13 in such a way to control the evaporator heater sensor 31 by receiving a signal from the evaporator heater sensor 32.

The enrichment apparatus 15 is connected with the evaporator 13 and the vacuum pump 16. In the interior of the enrichment apparatus 15 is provided an enrichment chamber 33 for accommodating hydrogen peroxide or moisture vaporized from the evaporator 13. Between the enrichment apparatus 15 and the evaporator 13 is provided an enrichment adjusting valve 34, and between the enrichment apparatus 15 and the vacuum pump 16 is disposed an enrichment chamber pressure adjusting valve 35.

At the enrichment apparatus 15 are installed the enrichment chamber pressure release valve 36 and the enrichment chamber pressure meter 37. The controller 47 controls the enrichment adjusting valve 74, the enrichment chamber pressure adjusting valve 35 and the enrichment chamber pressure release valve 36. The temperature of the enrichment chamber 33 is controlled by the enrichment chamber heater 38. The enrichment chamber heater 38 automatically operates by means of the controller 47. The controller 47 receives a signal from the enrichment chamber heater sensor 39 and controls the enrichment chamber heater 38, thus constantly maintaining the temperature of the enrichment chamber 33.

The vacuum pump 16 is controlled by means of the controller 47 and provides a vacuum pressure to the sterilization chamber 1, thus reducing the internal pressure of the sterilization chamber 11. In addition, the vacuum pump 16 serves to provide a vacuum pressure to the enrichment chamber 33, thus reducing the internal pressure of the enrichment chamber 33. When the pressure of the enrichment chamber 33 lowers and the enrichment adjusting valve 34 open, the moisture contained in the hydrogen peroxide provided to the evaporator 13 vaporizes and is sucked into the enrichment chamber 33.

As shown in FIG. 1, the blower 17 is formed of a fan 40 disposed in the interior of the sterilization chamber 11 and a motor 41 for the purpose of rotating the fan 40. The motor 41 is installed at an outer side of the housing 12, and the rotary shaft 41a of the motor 41 passes through the side wall of the housing 12 and is connected with the fan 40. As not shown, at a side wall of the housing 12 that the rotary shaft 41a of the motor 41 passes through is installed a sealing member for the purpose of preventing the communication with the sterilization chamber 11 by way of the flow of air. The surface of the fan 40 is coated with a photo catalyst 42 such as TiO₂.

The fan 40 is disposed close to the side wall of one side of the sterilization chamber 11, so the air in the sterilization chamber 11 can be more effectively circulated. A guide plate 43 is disposed at a front side of the fan 40 for the sake of smooth flow of air. The guide plate 43 has a passage 44 facing the fan 40. The fan 40 rotates in the rotation that the air of the sterilization camber 11 is pulled, and the air of the sterilization chamber 11 moves to the rear side of the fan 40 by way of the passage 44 of the guide plate 43 and the fan 40.

The air flowing to the rear side of the fan 40 is heated by means of the fan heater 24 disposed between the fan 40 and the side wall, and the air passed through the fan heater 24 collides with the side wall and flows to the outer side of the fan heater 24 along the side wall, and the air flowing to the fan heater 24 flows to the front side of the fan 40 by way of the space between the guide plate 43 and the upper surface of the sterilization chamber 11, the space between the guide plate 43 and the lower surface of the sterilization chamber 11 or the space between the guide plate 43 and the front and rear walls of the sterilization chamber 11. So, the whole air of the sterilization chamber 11 can be quickly heated. The sterilization chamber temperature sensor 51 configured to detect the internal temperature of the sterilization chamber 11 is arranged at the passage 44 of the guide plate 43.

In the present invention, the air of the sterilization chamber 11 can be heated using either the first sterilization chamber heater 25 or the second sterilization chamber heater 26 surrounding the sterilization chamber 11 without using the fan heater 24. In addition, the rotation direction of the fan 40 might be opposite to the above described direction.

As shown in FIG. 1, at one side of the sterilization chamber 11 is installed a violet ray generator 18. The violet ray generator 18 consists of a violet ray lamp 45 generating violet ray and a violet ray sensor 46 for detecting the violet ray. Here the violet ray lamp 45 and the violet ray sensor 46 are controlled by the controller 41. The controller 41 serves to control the operations of the violet ray lamp 45 in response to a signal from the violet ray sensor 46. The moisture and hydrogen peroxide vapor contained in the air can be converted into OH radicals using the photo catalyst 42 and the violet ray, thus enhancing the immunity along with the enhanced deodorizing effects. The detailed operations of the photo catalyst 42 and the violet ray will be described later. The photo catalyst 42 might be provided on the inner surface of the housing 12 in addition to the fan 40 or the guide plate 43 in the interior of the sterilization chamber 11.

As shown in FIG. 2, the controller 47 serves to control the whole operations of the sterilization apparatus 10 by receiving a control signal from various sensors and pressure meter and the input apparatus 48. The controller 47 can output the operation states of the sterilization apparatus 10, such as the temperature and pressure of the chamber 11 with the aid of the output apparatus 49. In the drawings, reference numeral 50 means a filter serving to prevent the input of the contaminated air into the sterilization chamber 11 when the sterilization chamber pressure release valve 22 is open.

The procedures of sterilizing the to-be-sterilized objects using the sterilization apparatus using hydrogen peroxide according to an embodiment of the present invention will be described with reference to the accompanying drawings.

The to-be-sterilized objects are washed, and the washed to-be-sterilized objects are inputted into the sterilization chamber 11. At this time, the door 19 is closed, and the motor 41 is driven under the atmospheric environment, and the fan 40 is rotated. At the same time, the fan heater 24 is operated. At this time, as the air in the interior of the sterilization chamber 11 starts flowing and is heated by the fan heater 24 and is uniformly spread to the to-be-sterilized objects and in the sterilization chamber 11. As the first sterilization chamber heater 25 and the second sterilization chamber heater 26 are operated along with the fan heater 24, the internal temperature and the to-be-sterilized objects in the sterilization chamber 11 can be more quickly raised. Here, the temperature of the sterilization chamber 11 is in a range of 3060° C. which can be adjustable.

In addition, the first sterilization can be implemented by means of the OH radicals in the middle of the above described processes. In a state that the temperature is raised, the violet ray generator 18 emits violet ray to the photo catalyst 42 coated on the fan 40, and with the air of reaction between the photo catalyst 42 and violet ray, the oxygen and moisture in the air can be converted, with the aid of the photo catalyst 42 and the violet rays, to a state of plasma in which oxygen ions such as singlet oxygen (01−), super oxide anion (02−), photo ozone (−03) and hydroxyl ion (OH−), hydrogen proton (H+) are mixed. In addition, it is circulated using the fan 40, and the generation and chain reaction are performed, thus generating a high density OH radical. So, the expensive plasma can be generated at a low cost in a simpler way. Here, the temperature of the sterilization chamber 11 can be adjusted to have a certain value within the above mentioned range. The first sterilization might be omitted if necessary depending on the process.

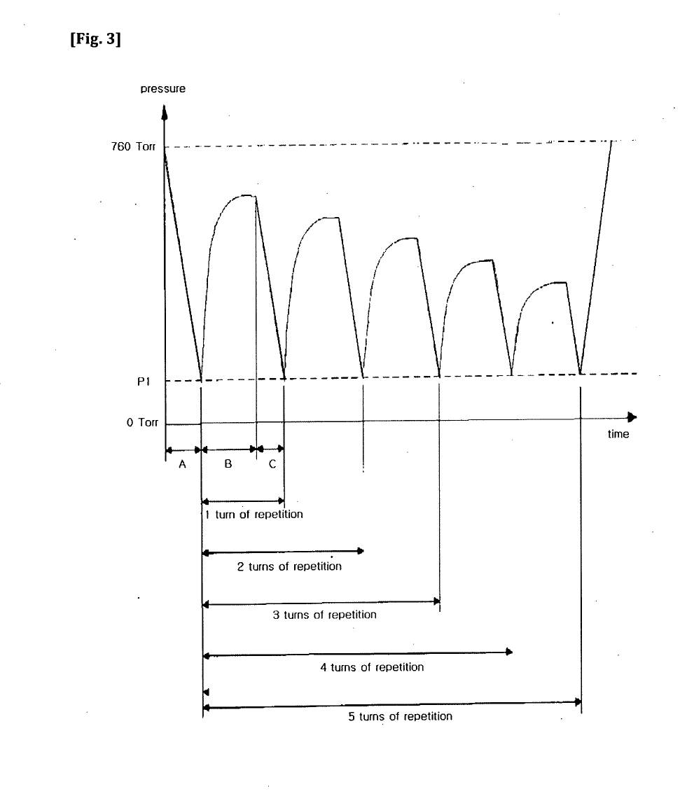

While the temperatures of the sterilization chamber 11 and the to-be-sterilized objects are being raised to the temperatures higher than room temperature (for example, 50° C.˜60° C.), hydrogen peroxide is enriched in the evaporator 13. The procedure of enriching hydrogen peroxide is as follows. First of all, the temperatures of the evaporator 13 and the enrichment chamber 33 are controlled to be same by using the evaporator heater 13 and the enrichment chamber heater 38, and liquid hydrogen peroxide is supplied to the evaporator 13 by adjusting the hydrogen peroxide supply adjusting valve 30. Next, the hydrogen peroxide supply adjusting valve 30 is closed, and the vacuum pump 16 is operated, and the enrichment chamber pressure adjusting valve 35 is open, so the pressure of the enrichment chamber 33 is lowered to a proper evaporation pressure P₁ below the atmospheric pressure. The above mentioned procedures are indicated by A in FIG. 3.

When the pressure of the enrichment chamber 33 drops to the evaporation pressure P₁, the enrichment adjusting valve 34 is open. At this time, the moisture contained in the hydrogen peroxide supplied to the evaporator 13 vaporizes and is sucked into the enrichment chamber 33. When moisture is inputted into the enrichment chamber 33, the pressure of the enrichment chamber 33 rises, and when moisture is saturated, the pressure of the enrichment chamber 33 is maintained constant, which procedures are indicated by B in FIG. 3.

The pressure rise of the enrichment chamber 33 owing to the input of moisture changes depending on the amount and concentration of the hydrogen peroxide supplied to the evaporator 13 and the volume, temperature and pressure of the enrichment chamber 33 and the temperature of the evaporator 13. When the moisture vaporizes and is introduced into the enrichment chamber 33, part of the hydrogen peroxide vaporizes and might be introduced into the enrichment chamber 33 along with moisture. When the moisture sucked into the enrichment chamber 33 is saturated, the enrichment adjusting valve 34 is closed.

Next, the enrichment chamber pressure adjusting valve 35 is open, and the moisture introduced into the enrichment chamber 33 is removed from the enrichment chamber 33 with the aid of the vacuum pump 16, and the pressure of the enrichment camber 33 is lowered to the evaporation pressure P₁ which procedure is indicated by C in FIG. 3, and the procedure B and C defines one enrichment turn. As the moisture is removed from the hydrogen peroxide supplied to the evaporator 13, the concentration of the hydrogen peroxide supplied to the evaporator 13 increases. As shown in FIG. 3, as the enrichment procedures are repeatedly performed, the internal pressure of the evaporator 13 drops, and the concentration of the hydrogen peroxide supplied to the evaporator 13 is continuously raised.

Here, the amount and concentration of the supplied hydrogen peroxide and the volume, temperature and pressure of the enrichment chamber 33 and the temperature of the evaporator 13 might change depending on the kinds, volume and number of the to-be-sterilized objects in many ways. The concentration of the supplied hydrogen peroxide is in a range of 1 wt %˜60 wt %, and the temperature of the evaporator 13 is in a range of 30° C.˜150° C. In addition, the enrichment chamber 33 has a size of 10 ml˜the volume of sterilization chamber, and the temperatures is in a range of 30° C.˜150° C. The evaporation pressure P₁ of the enrichment chamber 33 is a proper value between 0˜760 Torr below the atmospheric pressure.

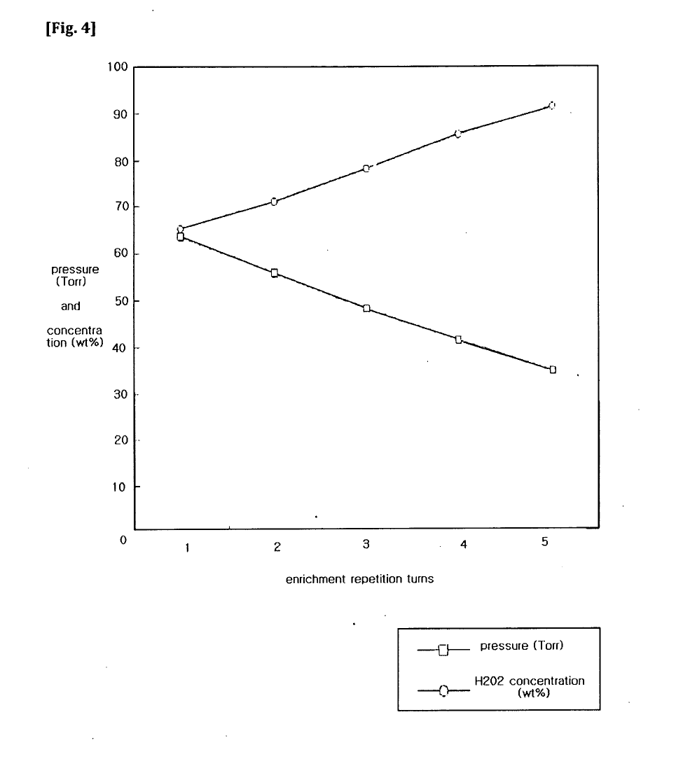

The table 1 and FIG. 4 show a result of the experiments which were performed five turns as show in FIG. 2 with respect to the above described enrichment procedures.

TABLE 1

| | | | 1 | | 2 | | 3 | | 4 | | 5 | |
|---|---|---|---|---|---|---|---|---|---|---|---|---|
| | 0 turn | | turn | | turns | | turns | | turns | | turns | |
| Amount of hydrogen peroxide | 5 | ml | 5 | ml | 5 | ml | 5 | ml | 5 | ml | 5 | ml |
| Concentration of hydrogen | 58 | wt % | 58 | wt % | 58 | wt % | 58 | wt % | 58 | wt % | 58 | wt % |
| peroxide | | | | | | | | | | | | |
| Temperature of evaporator | 60 | | 60 | | 60 | | 60 | | 60 | | 60 | |
| Temperature of enrichment | 60 | | 60 | | 60 | | 60 | | 60 | | 60 | |
| chamber | | | | | | | | | | | | |
| Start pressure of enrichment | 5 | Torr | 5 | Torr | 5 | Torr | 5 | Torr | 5 | Torr | 5 | Torr |
| chamber | | | | | | | | | | | | |

| | | | | | | | | | | | |
|---|---|---|---|---|---|---|---|---|---|---|---|
| End pressure of enrichment | — | 64 | Torr | 56 | Torr | 48 | Torr | 41 | Torr | 34 | Torr |
| chamber | | | | | | | | | | | |
| Amount of residual hydrogen | — | 4.3 | ml | 3.6 | ml | 2.9 | ml | 2.2 | ml | 1.6 | |
| peroxide | | | | | | | | | | | |
| Concentration of residual | — | 65 | wt % | 71 | wt % | 78 | wt % | 85 | wt % | 91 | wt % |
| hydrogen peroxide | | | | | | | | | | | |

In the present experiments, the amount of the hydrogen peroxide used for sterilization is 5 ml, and the concentration of hydrogen peroxide is 58 wt %, and the volume of the enrichment chamber 33 is 5 liter, and the temperature is 60° C., and the capacity of the evaporator is 100 ml, and the temperature is set at 60° C., and the evaporation pressure P₁ of the enrichment chamber 33 is 5 Torr.

Referring to Table 1 and FIG. 4, as the enrichment procedures are repeatedly performed, the internal pressures of the evaporator 13 and the enrichment chamber 33 constantly lower, and the concentration hydrogen peroxide supplied to the evaporator 13 continuously rises. Here, since the enrichment adjusting valve 34 is open, the pressures of the evaporator 13 and the enrichment chamber 33 are same.

The following experiments were conducted so as to check the enhancement effects of the sterilization power by way of the enrichment of hydrogen peroxide.

In the middle of the procedures that the enrichment is performed by way of the enrichment chamber 33, the sterilization chamber pressure adjusting valve 21 is open, and the pressure of the sterilization chamber 11 is lowered to about 0.5˜1 Torr. The pressure of the sterilization chamber 11 is less than the atmospheric pressure and preferably, it can be adjusted to be a proper valve below 100 Torr. The pressure adjustment of the sterilization chamber 11 might be performed at the same time together with the enrichment procedure, and it might be performed after the enrichment or before it. The pressure of the sterilization chamber 11 is preferably a vacuum state lower than the atmospheric pressure when the enriched hydrogen peroxide is injected.

When the above mentioned hydrogen peroxide enrichment procedure is completed, the enrichment adjusting valve 34 is closed, and the hydrogen peroxide vapor supply valve 20 is open, and the enriched hydrogen peroxide is supplied to the sterilization chamber 11 and the sterilization procedure on the to-be-sterilized objects in the sterilization chamber 11 is performed. In addition, the enrichment chamber pressure adjusting valve 35 is closed, and the enrichment chamber pressure release valve 36 is open, so the enrichment chamber 33 returns to the atmospheric state.

In the present experiment, as the biological indicator used to check the effects of sterilization, 10⁶ numbers of Bacillus Stearothermophilus viable sores were used. As the to-be-sterilized objects accommodated in the sterilization chamber 11, 10 scissors for surgery in which living fungi were cultivated in a hinge which is hard to penetrate in and 10 hard lumen in which living fungi were cultivated at the center of φ1 mm×60 cm of a stainless pipe representing a hard endoscope, 10 soft lumen in which living fungi were cultivated at the center of φ1 mm×2 mm of the teflon pipe representing a soft endoscope and 5 PCDs in which a disc with a living fungus being cultivated is inserted into an end portion of an inner diameter of Φ2×1,500 mm which is closed at its one side and is generally used as PCD (Process Challenge Device) of an oxide ethylene gas sterilizer and a vacuum type steam sterilizer were used, so the sterilization performances were compared and evaluated. At this time, the set temperature of the sterilization chamber 11 is 50° C., and the capacity of the sterilization chamber 11 is 50 liter.

Result of the above described experiments is shown in Table 2.

TABLE 2

| | 1 turn of | 2 turns of | 3 turns of | 4 turns of | 5 turns of |
|---|---|---|---|---|---|
| | enrichment | enrichment | enrichment | enrichment | enrichment |
| Scissor for | 5/10 | 7/10 | 10/10 | 10/10 | 10/10 |
| surgery | | | | | |
| Hard Lumen | 0/10 | 3/10 | 7/10 | 9/10 | 10/10 |
| (Φ1 mm × 600 mm) | | | | | |
| Soft Lumen | 12/10 | 5/10 | 8/10 | 10/10 | 10/10 |
| (Φ1 mm × 2,000 mm) | | | | | |
| One side-closed PCD | 0/5 | 0/5 | 2/5 | 4/5 | 5/5 |
| (Φ2 mm × 1,500 mm) | | | | | |

As shown in Table 2, as the turns of the enrichment procedures increase, the sterilization effects are enhanced. As the turns of the repetition of the enrichment procedures increase, the amount of hydrogen peroxide reduces; however the concentration increases, so the penetration power of hydrogen peroxide is enhanced. In other words, as the concentration of hydrogen peroxide increases, the partial pressure drops relatively as compared to the hydrogen peroxide, so it is possible to prevent the moisture with lower molecular weight from reaching the portions of the to-be-sterilized objects, so the problems that the introduction of hydrogen peroxide is interrupted can be reduced, and consequently the sterilization power can be enhanced.

Continuously, the sterilization procedures using the sterilization apparatus using hydrogen peroxide according to an embodiment of the present invention will be described.

When the hydrogen peroxide enrichment procedures is completed to the extent that the to-be-sterilized objects in the sterilization chamber 11 can be sterilized, and the temperature of the to-be-sterilized objects rises, the enrichment adjusting valve 34 is closed, and the hydrogen peroxide vapor supply valve 20 is open, and the enriched hydrogen peroxide is supplied to the sterilization chamber 11.

While the hydrogen peroxide is being supplied, the pressure of the sterilization chamber 11 is adjusted to have a proper vacuum pressure by opening the sterilization chamber pressure adjusting valve 21. The pressure of the sterilization chamber 11 is less than the atmospheric pressure, preferably, can be adjusted to be lower than 100 Torr. When the hydrogen peroxide vapor introduced into the sterilization chamber 11, the fan 34 and the violet ray generator 17 do not work.

When hydrogen peroxide is injected, the pressure of the sterilization chamber 11 rises. When it becomes saturated, it keeps constant. In the above process, the hydrogen peroxide vapor penetrates into the to-be-sterilized objects. Afterward, the pressure of the sterilization chamber 11 rises by opening the sterilization chamber pressure release valve 22, so further penetrations of the hydrogen peroxide vapor are performed with respect to the to-be-sterilized objects, so that the second sterilization is performed to further sterilize the to-be-sterilized objects with hydrogen peroxide vapor

Under the atmospheric pressure, the violet ray lamp 45, the fan 40 and the fan heater 24 are operated, and a plasma state is obtained, thus generating OH radicals and performing the third sterilization. In the middle of the above operations, hydrogen peroxide is decomposed into water and oxygen, and in the middle of the generation of the OH radicals, the decomposition and deodorization of hydrogen peroxide are performed. Afterward, the sterilization chamber 11 is vacuumed for the purpose of eliminating the remaining hydrogen peroxide, and external air is supplied again, thus completing the sterilization process.

Since the sterilization chamber 11 and the to-be-sterilized objects are heated using the blower 17 and the fan heater 24, the phenomenon that hydrogen peroxide vapor is condensed is prevented, so more hydrogen peroxide vapor can penetrate into the to-be-sterilized objects with the aid of the above mentioned operations, thus enhancing the sterilization effects.

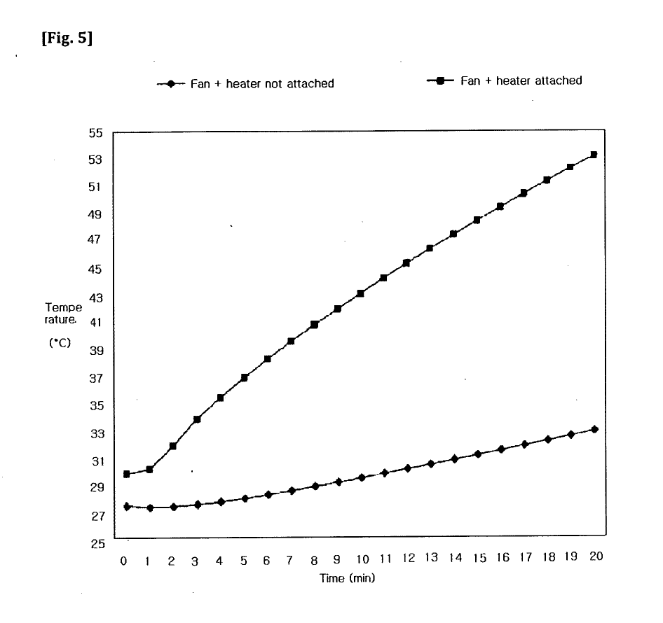

The graph of FIG. 5 shows a comparison of the temperature change of the sterilization chamber as time passes when the fan and the fan heater are not used and when they are used. As shown in FIG. 5, when the fan 40 and the fan heater 24 are used, it is possible to quickly raise the temperature of the sterilization chamber. Here, the volume of the sterilization chamber 11 used in the experiment is 50 liter, and the capacity of the blower 17 is 15˜20 m³/min, and the capacity of the fan heater 24 is 1500 W.

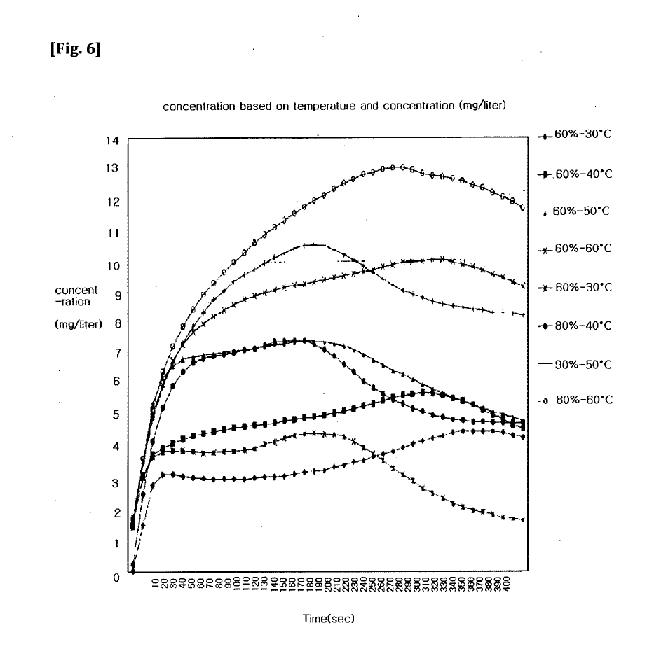

The graph of FIG. 6 shows a result of the measurement of the hydrogen peroxide concentration in the interior of the sterilization chamber based on the temperature of the sterilization chamber and the concentration of the hydrogen peroxide. As shown in FIG. 6, it seems that the concentrations of 60 wt % of 50° C. and 80 wt % of 40° C. are same, which shows that the rise of the temperature has as much effects as when the hydrogen peroxide with high concentration is used.

The sterilization apparatus 10 using hydrogen peroxide according to an embodiment of the present invention is directed to enhancing sterilization efficiencies by using the photo catalyst 42 coated on the fan 40 and the violet ray generator 18. The operations of the photo catalyst 42 and the violet rays are as follows.

In a state that the hydrogen peroxide is introduced into the interior of the sterilization chamber 11, when the violet ray generator 18 emits violet ray to the photo catalyst 42 coated on the fan 40, the oxygen and moisture and hydrogen peroxide in the air can be converted, with the aid of the photo catalyst 42 and the violet rays, to a state of plasma in which oxygen ions such as singlet oxygen (01−), super oxide anion (02−), photo ozone (03−) and hydroxyl ion (OH−), hydrogen proton (H+) are mixed. In addition, it is circulated using the fan 40, and the generation and chain reaction are performed, thus generating a high density OH radical. So, the expensive plasma can be generated at a low cost in a simpler way.

The oxidation power of OH radical is known to have sterilization power which is about 1.35 times higher than ozone, and about 1.86 times higher than chlorine dioxide and about 1.57 times higher than hydrogen peroxide. So, as compared to the sterilization method of using only hydrogen peroxide, it is possible to more enhance the sterilization power by using the plasma before and after the sterilization is performed using hydrogen peroxide.

The following table 3 shows a result of the comparison experiment of the sterilization power when the fan, the fan heater and the violet ray generator do not operate and they are operated.

TABLE 1
(extinction rate/experiment rate)

| | fan, fan heater | |
|---|---|---|
| | and violet ray | fan, fan heater |
| | generator do | and violet ray |
| | not operate | generator operate |
| Scissor for | 15/10 | 10/10 |
| surgery | | |
| Hard Lumen | 0/10 | 9/10 |
| (φ1 mm x 600 mm) | | |
| Soft Lumen | 2/10 | 10/10 |
| (φ1 mm × 2,000 mm) | | |
| One side-closed PCD | 0/5 | 4/5 |
| (φ2 mm × 1,500 mm) | | |

In the present experiment, as a biological indicator system for checking the sterilization effects in the present experiments, 10⁶ number of Bacillus Stearothermophilus viable spore were used. As the to-be-sterilized objects accommodated in the sterilization chamber 11, 10 scissors for surgery in which living fungi were cultivated in a hinge which is hard to penetrate in and 10 hard lumen in which living fungi were cultivated at the center of φ1 mm×60 cm of a stainless pipe representing a hard endoscope, 10 soft lumen in which living fungi were cultivated at the center of φ1 mm×2 mm of the teflon pipe representing a soft endoscope and 5 PCDs in which a disc with a living fungus being cultivated is inserted into an end portion of an inner diameter of φ2×1,500 mm which is closed at its one side and is generally used as PCD (Process Challenge Device) of an oxide ethylene gas sterilizer and a vacuum type steam sterilizer were used, so the sterilization performances were compared and evaluated when the fan 40, the fan heater 24 and the violet ray generator 18 were operated and when they were not operated.

In the present experiment, the volume of the sterilization chamber 11 was 50 liter, the temperature of the sterilization chamber 11 was 50° C., and the capacity of the blower 17 was 15˜20 m³/min, and the capacity of the fan heater 24 was 1500 W, and the concentration of the supplied hydrogen peroxide was 2 ml for 58 wt %, and the emitting level of the violet ray was 20 W, and the photo catalyst 42 as TiO₂, and the surface area of the photo catalyst 42 was about 40 cm². The above described experiments are aimed at evaluating the effects of the fan, the fan heater and the violet ray generator, so there is provided a result of the experiment conducted using the supplied hydrogen peroxide without performing the enrichment procedures of the hydrogen peroxide.

The sterilization performance and the decomposition and deodorization effects of the hydrogen peroxide can be enhanced using the photo catalyst 42 and the violet ray. In the present invention, since the sterilization by plasma is performed in an atmospheric state, not in a vacuum state, the hydrogen peroxide can be decomposed in the principle below, and various smells occurring in the middle of sterilization and the inherent smell of the hydrogen peroxide can be decomposed and deodorized.

(1) As violet rays are emitted to the photo catalyst 42 (TiO₂), electrons (e-) and positive holes generate.

(2) The generated electrons and positive holes react with oxygen, water and hydrogen peroxide and superoxide radical (02−) and hydroxy radical (OH++) are generated.

H₂O₂→H₂O+O₂

O₂+e−=O₂−(+)H₂O+h+=OH+

(3) The generated radicals (O₂−, OH+) have strong oxidation powder, thus oxidizing and reducing bad smell, virus, bacteria, fungus, NOx, VOCs.

(4) The above mentioned reactions are performed in chain and repeatedly using the fan 40, thus converting to CO₂ and H₂O.

By way of the above mentioned procedures, hydrogen peroxide is decomposed, and the inherent smell of hydrogen peroxide as well as various bad smells generating in the middle of sterilization can be decomposed and deodorized.

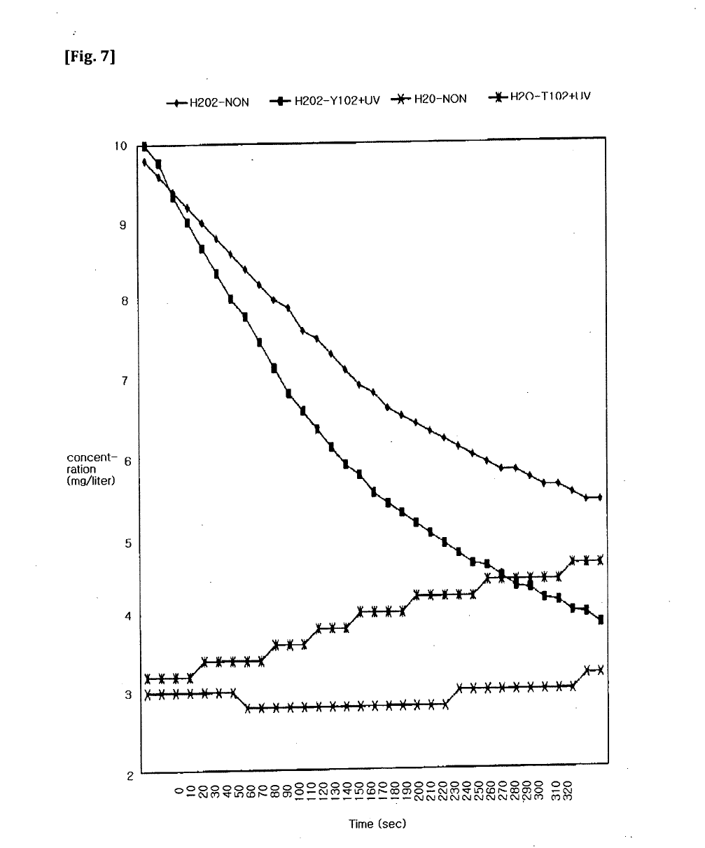

FIG. 7 is a graph showing the concentration changes of hydrogen peroxide measured every 10 seconds while comparing them before and after the fan and the violet ray generator are operated. As shown in FIG. 7, when comparing the concentrations of hydrogen peroxide and water of the sterilization chamber 11 when the photo catalyst 42 and the violet ray are used and when they are not used, the speed that the hydrogen peroxide becomes water is much faster when the photo catalyst and violet ray are scanned.

In the examples of the experiments, the temperature of the sterilization chamber 11 was 50° C., and the capacity of the sterilization chamber 11 was 50 liter, and the emitting level of the violet ray was 20 W, and the coated surface area of the photo catalyst 42 was about 40 cm², and the capacity of the blower 17 was 15˜20 m³/min, and the supplied hydrogen peroxide was 2 ml with respect to the concentration 58 wt %.

The sterilization apparatus using hydrogen peroxide according to an embodiment of the present invention has a drying function of drying to-be-sterilized objects. Drying the to-be-sterilized objects is as follows.

When the sterilization process is finished, the temperatures of the to-be-sterilized objects and the remaining moistures in the interior of the sterilization chamber 11 are raised using the fan 40 and the fan heater 24 under atmospheric environment. At this time, the heating temperature is 3060° C. When the to-be-sterilized object is heated up to a proper temperature, the sterilization chamber pressure adjusting valve 21 is opened, and the vacuum pump 16 is operated, thus vacuuming, so the pressure of the sterilization chamber 11 is dropped to a certain level. At this time, the pressure of the sterilization chamber 11 is lower than atmospheric level, preferably lower than 100 Torr.

The sterilization chamber 11 is vacuum-exhausted for a certain time. If it is not fully vacuum-exhausted, the sterilization chamber pressure adjusting valve 21 is closed, and the sterilization chamber pressure release valve 22 is open, and air is injected again into the sterilization chamber 11. In addition, the fan 40 and the fan heater 24 are operated, and the temperature of the interior of the sterilization chamber 11 is raised, and then the vacuum exhaust process is performed. With the above mentioned heating and vacuum-exhaust processes are repeatedly performed, the moisture remaining in the to-be-sterilized objects can be fully eliminated.

The Table 4 below is a result of the experiments showing the sterilization power enhancement effects when the fan 40, the fan heater 24 ad the violet ray generator 18 operate along with the enrichment of the hydrogen peroxide.

The table 4 below is a result of the experiments after the sterilization power is compared when the fan, the fan heater and the violet ray generator do not operate in a state that the hydrogen peroxide is not enriched and when they work in a state that the hydrogen peroxide is enriched.

TABLE 4
(extinction amount/experimental amount)

| | When fan, | | | | | |
|---|---|---|---|---|---|---|
| | fan heater | | | | | |
| | and violet ray | | | | | |
| | generator do | | | | | |
| | not operate | When fan, fan heater and violet ray generator operate | | | | |
| | Not enriched | 1 turn of | 2 turns of | 3 turns of | 4 turns of | 5 turns of |
| | (58 wt %) | enrichment | enrichment | enrichments | enrichment | enrichment |
| Hard | 0/10 | 3/10 | 5/10 | 8/10 | 10/10 | 10/10 |
| Lumen(φ0.5 mm × | | | | | | |
| 600 mm) | | | | | | |
| Soft | 0/10 | 4/10 | 7/10 | 9/10 | 10/10 | 10/10 |
| Lumen(φ0.5 mm × | | | | | | |
| 2,000 mm) | | | | | | |

In the present experiment, as the biological indicator used to check the effects of sterilization, 10⁶ numbers of Bacillus Stearothermophilus viable sores were used. As the to-be-sterilized objects accommodated in the sterilization chamber 11, 10 hard lumen in which living fungi were cultivated at the center of 10.5 mm×60 cm of a stainless pipe representing a hard endoscope, 10 soft lumen in which living fungi were cultivated at the center of 10.5 mm×2 mm of the teflon pipe representing a soft endoscope. As a result of the experiments, the sterilization power can be greatly enhanced when the hydrogen peroxide is enriched and supplied to the sterilization chamber 11, and the fan 40, the fan heater 24 and the violet ray generator 18 are used.

The sterilization apparatus 10 using hydrogen peroxide according to an embodiment of the present invention makes it possible to easily control the amount and concentration of hydrogen peroxide depending on the types of the to-be-sterilized objects such as medical tools, etc., thus enhancing the sterilization efficiency of the to-be-sterilized objects. For example, simple looking to-be-sterilized objects can be sterilized for short time using a small amount of hydrogen peroxide, and higher sterilization power can be obtained by prolonging the whole work time with respect to complicated to-be-sterilized objects and raising the concentration of the hydrogen peroxide. In addition, it is possible to reduce the potential dangers that the to-be-sterilized objects are damaged, in such a way that the hydrogen peroxide of a low concentration is supplied to the to-be-sterilized objects which are weak to hydrogen peroxide.

In addition, as the heated wind is supplied using the fan 40 and the fan heater 24, the sterilization power with respect to the to-be-sterilized objects can be enhanced, and the to-be-sterilized objects can be dried using a separate drier.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A sterilization apparatus using hydrogen peroxide, comprising:
a housing which has a sterilization chamber for accommodating to-be-sterilized objects;
an evaporator connected with the sterilization chamber for supplying hydrogen peroxide vapor to the sterilization chamber for the purpose of sterilizing to-be-sterilized objects;
a hydrogen peroxide supply apparatus connected with the evaporator for the purpose of supplying liquid hydrogen peroxide to the evaporator;
an enrichment chamber connected with the evaporator for the purpose of evaporating moisture from the hydrogen peroxide supplied to the evaporator and sucking the evaporated moisture, thus enriching the hydrogen peroxide;
a vacuum pump connected with the enrichment chamber for the purpose of providing a vacuum pressure to the enrichment chamber;
a blower which includes a fan disposed in the interior of the sterilization chamber for forcibly flowing the air of the sterilization chamber and a motor for driving the fan; and
a fan heater disposed in the interior of the sterilization chamber for the purpose heating the air flowing by means of the fan,
wherein the fan is disposed closer to a side wall of one side of the housing, and the motor is disposed at an outer side of the side wall, and a rotary shaft of the motor passes through the side wall and is connected with the fan, and the fan heater is disposed between the fan and the side wall, and at the fan is installed a guide plate which faces the fan at the front side of the fan in the interior of the sterilization chamber and which rotates in a rotation direction that the air flowing to the fan gathers at the center of the fan, and the air of the interior of the sterilization chamber collides with the side wall by way of the fan and the fan heater and flows to the front side of the fan by way of the space between the guide plate and the inner surface of the housing.

2. A sterilization apparatus using hydrogen peroxide according to claim 1, further comprising an enrichment adjusting valve which is disposed between the evaporator and the enrichment chamber for thereby closing and opening the flow passage between the evaporator and the enrichment chamber.

3. A sterilization apparatus using hydrogen peroxide according to claim 1, further comprising an enrichment chamber pressure release valve connected to the enrichment chamber for introducing an external air into the interior of the enrichment chamber.

4. A sterilization apparatus using hydrogen peroxide according to claim 1, wherein the vacuum pump is connected with the sterilization chamber for providing a vacuum pressure to the sterilization chamber, and between the sterilization chamber and the vacuum pump is disposed a sterilization chamber pressure adjusting valve for opening and closing the flow passage between the sterilization chamber and the vacuum pump.

5. A sterilization apparatus using hydrogen peroxide according to claim 1, further comprising a sterilization chamber pressure release valve connected with the sterilization chamber for introducing an external air into the interior of the sterilization chamber.

6. A sterilization apparatus using hydrogen peroxide according to claim 1, further comprising an evaporator heater for heating the evaporator.

7. A sterilization apparatus using hydrogen peroxide according to claim 1, further comprising an enrichment chamber heater for heating the enrichment chamber.

8. A sterilization apparatus using hydrogen peroxide according to claim 1, wherein the volume of the enrichment chamber is a size in a range from 10 ml˜the volume of the sterilization chamber.

9. A sterilization apparatus using hydrogen peroxide according to claim 1, further comprising an enrichment chamber pressure meter connected with the enrichment chamber for detecting the pressure of the enrichment chamber.

10. A sterilization apparatus using hydrogen peroxide according to claim 1, further comprising a sterilization chamber temperature sensor disposed at the passage for detecting the temperature of the interior of the sterilization chamber.

11. A sterilization apparatus using hydrogen peroxide according to claim 1, further comprising:
a photo catalyst disposed in the interior of the sterilization chamber; and
a violet ray generator which has a violet ray lamp disposed in the interior of the sterilization chamber for emitting violet ray to the photo catalyst.

12. A sterilization apparatus using hydrogen peroxide according to claim 11, wherein the photo catalyst is coated on at least one of the surface of the fan, the inner surface of the housing and the guide plate.

13. A sterilization apparatus using hydrogen peroxide according to claim 11, wherein the photo catalyst contains TiO₂.
